(19) United States
(12) Patent Application Publication
(10) Pub. No.: US 2004/0115697 A1
(43) Pub. Date: Jun. 17, 2004

(54) Cancer diagnostics and prognostics

(76) Inventors: Stephen J. Doxsey, Princeton, MA (US); German Pihan, Weston, MA (US)

Correspondence Address: FISH & RICHARDSON PC, 225 FRANKLIN ST, BOSTON, MA, 02110, US

(21) Appl. No.: 10/639,812

(22) Filed: Aug. 11, 2003

Related U.S. Application Data
(60) Provisional application No. 60/402,435, filed on Aug. 9, 2002.

(51) Int. Cl. C12Q 1/68; G01N 1/30; G01N 33/48

(52) U.S. Cl. 435/6; 435/40.5

(57) ABSTRACT

The invention is based on the discovery that occurrence of centrosomal abnormalities in cells correlates with the occurrence of cancer, and that the greater the degree of the centrosomal abnormalities, the greater the probability of cancer occurring and the severity of the cancer. The invention includes methods of detecting centrosome abnormalities in tissue samples. It provides new methods for predicting and diagnosing cancer.

### CROSS-REFERENCE TO RELATED APPLICATIONS

This application claims priority from U.S. Provisional Patent Application No. 60/402,435, filed on Aug. 9, 2002, which is incorporated herein by reference in its entirety.

### FIELD OF THE INVENTION

This invention relates to methods of predicting and diagnosing cancer.

### BACKGROUND OF THE INVENTION

Cancer is a category of related diseases in which normal, healthy cells become cancerous cells. Normally, cells grow and divide in a relatively orderly manner to produce more cells only when required by the body. In cancer, however, cells continue to divide and proliferate even when new cells are not required. This can lead to the formation of a mass of tissue, such as a growth or tumor. Cancer is one of the leading causes of death worldwide. Prostate, breast, and cervical cancer are among the most prevalent forms of cancer, and cause many deaths.

Centrosomes play critical roles in processes that affect the genetic stability of human cells. They are involved in mitotic spindle organization, cytokinesis and cell cycle progression, processes essential for ensuring the fidelity of chromosome segregation. Centrosomes are the primary microtubule-organizing centers in animal cells and they contribute to the organization of microtubule spindles in mitosis and control progression through cytokinesis and entry into S phase.

### SUMMARY OF THE INVENTION

The invention is based, in part, on the discovery that occurrence of centrosomal abnormalities in cells correlates with the future occurrence of cancer. Thus, the invention provides new methods for predicting and diagnosing cancer, as well as providing a prognosis for the severity of a given tumor.

The invention features methods of predicting the evolution of an in situ lesion in a patient by examining a microtubule organizing center of a cell in a tissue sample (e.g., prostate, breast, uterine cervix, brain, lung, colon, or any other tissue in which carcinomas can occur) from the in situ lesion of the patient, detecting a centrosome abnormality in the cell, and determining the degree of severity of any centrosome abnormality detected, in which the degree of severity of any centrosome abnormality correlates with the probability that the in situ lesion will evolve into a high grade invasive cancer. These methods, and any other methods of the invention, can be entirely or partially automated.

The invention also features methods of predicting cancer in a patient by examining a microtubule organizing center of a cell in a tissue sample (e.g., prostate, breast, uterine cervix, brain, lung, colon, or any other tissue in which carcinomas can occur) from the patient, and detecting a centrosome abnormality (e.g., a diameter of a centrosome greater than twice the diameter of centrosomes present in normal epithelium in the same tissue sample, a centrosome in which the ratio of the centrosome's greatest and smallest diameter exceeds about 2, abnormal shape, absence of a centrosome, or centrosomes that are organized as multiple small dots, increased level of pericentrin) in the cell, in which the presence of a centrosome abnormality indicates an increased probability that the patient will develop cancer. This method can be repeated for multiple cells, in which case, the centrosome abnormality detected is the presence of more than two centrosomes in more than about 5% of the cells whose microtubule organizing centers are examined or in which or the ratio of centrosomes to nuclei is greater than about 2.5.

In another aspect, the invention encompasses methods of predicting the degree of aggressiveness of a cancer in a patient by examining a microtubule organizing center of a cell in a tissue sample (uterine cervix, breast, prostate, or any other tissue in which carcinomas can develop) from a precancerous lesion of the patient, detecting a centrosome abnormality in the cell, and determining the degree of severity of any centrosome abnormality detected, in which the degree of severity of any centrosome abnormality correlates with the probability that the patient has or will develop aggressive cancer (e.g., an approximately 2- to 4-fold increase in the incidence of centrosomal abnormality compared to normal cells correlates with histologic/cytologic grade of cancer).

The invention also encompasses methods of predicting cancer in a patient by examining a mitotic spindle of a cell in a tissue sample (e.g., uterine cervix, breast, prostate, or any other type of tissue in which carcinoma can develop) from the patient, and detecting any mitotic spindle abnormality in the cell, wherein detection of a mitotic spindle abnormality indicates an increased probability that the patient has or will develop cancer.

In addition, the invention includes methods of predicting cancer in a subject, in which the method includes measuring the level of pericentrin in a cell culture or tissue sample of interest, comparing the level of pericentrin in the cell culture or tissue sample of interest to the concentration of pericentrin in a normal, healthy control cell culture or tissue sample, and predicting an enhanced probability of developing cancer if the level of pericentrin in a cell culture or tissue sample of interest is greater (e.g., at least about twice) than that in the normal, healthy control cell culture or tissue sample.

Also, the invention features systems for detecting centrosome abnormalities automatically, in which the system includes a cell culture or tissue sample to be examined, a means for automatically preparing the cell culture or tissue sample (e.g., immunohistochemistry, immunofluoresence, paraffin-embedding of multiple samples) for examination, a high magnification microscope, an XY stage adapted for holding a plate containing a cell culture or tissue sample and having a means for moving the plate for proper alignment and focusing on the cell culture or tissue sample arrays, a digital camera, a light source having optical means for directing excitation light to cell culture or tissue sample arrays and a means for directing fluorescent light emitted from the cells to the digital camera, a computer means for receiving and processing digital data from the digital camera, wherein the computer means includes a digital frame grabber for receiving the images from the camera, a display for user interaction and display of assay results, digital storage media for data storage and archiving, and a means for control, acquisition, processing, and display of results, and a computer means for detecting centrosome abnormalities in the cell culture or tissue sample.

As used herein, “evolution” of cells refers to Darwinian selection for cells that have increased proliferation, increased survivability, and increased resistance to chemotherapy.

As used herein, “development” of cells or tissues or tumors refers to their progression through the stages of healthy to preinvasive to low, medium, and high (or aggressive) grades of cancer (e.g., as measured by the Gleason scale, the changes used to describe the aggressive of cells in a Pap smear or in indications of breast cancer, or the various scales or measuring units employed to measure severity, development, or progression of any carcinomas).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention provides a number of advantages. It allows the early prediction and diagnosis of cancer from tissue samples. This can enhance patient survivorship by allowing treatment for cancer to commence earlier than it would otherwise. This is particularly true with respect to three of the most common cancers: prostate, breast, and cervical. The invention also provides specific diagnostic features of centrosome abnormalities, thus enhancing the efficiency and accuracy of cancer prediction and diagnosis. Furthermore, it allows the determination of a prognosis about the severity of a particular cancer (e.g., prostate cancer), thus allowing treatment decisions (e.g., decision to elect surgery if prognosis is for aggressive cancer) to be made earlier than would otherwise be possible.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A-F are a series of micrographs that illustrate centrosome defects in carcinoma in situ. Photomicrographs (1000×) of normal epithelium (1A, 1C, and 1E) and in situ carcinoma (1B, 1D and 1F) immunostained with antibodies to pericentrin to visualize centrosomes. In normal epithelia, centrosomes are round and uniform in size (arrowheads, 1A, 1C and 1E) while in carcinoma in situ they are larger (arrowheads in 1B, 1D, 1F), multiple (1B), or structurally abnormal (arrowheads in 1D and 1F). Nuclei are stained light blue with hematoxylin. The inset in 1D shows higher magnification of an elongated centrosome.

FIGS. 2A-C are a series of graphs that illustrate that centrosome defects are prevalent in carcinoma in situ. Centrosome defects are present in 62, 75 and 28 percent of CIC (2A), DCIS (2B) and PIN (2C) lesions, respectively (N, normal epithelia). First column (2A-C), cumulative defects; second column (A′-C′), breakdown of centrosome defects by category (#, number, Sz, size, Sh, shape).

FIGS. 3A-L are a series of graphs that illustrate that the incidence of centrosome defects increases with increasing histologic grade. The cumulative incidence of centrosome defects in each pre-invasive lesion (left column) includes grades 1-3 for CIC (3A, 1-3) and low (3L) and high (3H) grades for DCIS (3E) and PIN (3I). N identifies normal epithelium. Each subcategory of centrosome defects increases with grade including increased centrosome number (3B, 3F, 3J), shape abnormalities (3C, 3G, 3K), and size (3D, 3H, 3L).

FIGS. 4A-I are a series of photomicrographs (at left) and graphs (at right) that illustrate that mitotic spindle defects are common in CIC and DCIS. Examples of bipolar mitotic spindles immunostained with g-tubulin in CIC and DCIS (4A and 4C, respectively). Examples of multipolar spindles (4B, CIC; 4D, 4F, DCIS) and multiple spindles (4E, DCIS). Quantitative analysis of the number of bipolar spindles (x axis) and mulitipolar spindles (y axis) in each CIC lesion (4G) and DCIS lesion (4H). Each circle represents a single lesion. Filled circles represent lesions with ten or more mitoses and were included in the estimation of the extent of mitotic spindle defects in CIC and DCIS. On average 10% and 17% of the spindles, in CIC and DCIS lesions with more than 10 immunostained spindles (red circles in 4G and 4H), are abnormal.

FIGS. 5A-I are a series of photomicrographs (at left) and graphs (at right) that illustrate that centrosome abnormalities correlate with chromosome instability in carcinoma in situ. Examples of in situ hybridization reactions performed on samples of CIC (5B), DCIS (5D) and PIN (5F). Many cells have more than two signals for chromosome #8 (arrowheads in 5B, 5D, 5F) and thus exhibit chromosome instability (CIN+). Cells in adjacent normal epithelium (5A, 5C, 5E) rarely have more than two signals. Quantitative analysis of chromosomal instability (CIN+) in CIC (5G), DCIS (5H) and PIN (5I) lesions with normal centrosomes (5N) or abnormal centrosomes (5A). CIN is present in most lesions with abnormal centrosomes and a small fraction of lesions lacking centrosome abnormalities.

FIGS. 6A-J are a series of photomicrographs (at top) and graphs (at bottom) that illustrate centrosome and spindle defects and chromosome instability in cell lines derived from in situ lesions. Immunofluorescence images showing centrosomes and spindles in cell lines derived from normal epithelium (1560NPTX, 6A, 6B, mitosis, 6E, interphase) and high grade PIN lesion from the same prostate gland (1560PINTX, 6C, 6D, mitosis, 6F, 6G, interphase). Quantification of this data shows that 1560PINTX has a 2-4-fold higher incidence of centrosome defects (6H), spindle defects (6I) and chromosome instability (6J) than 1560NPTX.

FIG. 7 is a schematic diagram depicting a centrosome-mediated model for tumor progression.

FIG. 8 is a diagram of the components of a cell-based scanning system. An inverted fluorescence microscope is used 1, such as a Zeiss Axiovert inverted fluorescence microscope that uses standard objectives with magnification of 1-100× to the camera, and a white light source (e.g., 100W mercury-arc lamp or 75W xenon lamp) with power supply 2. There is an XY stage 3 to move the plate 4 in the XY direction over the microscope objective. A Z-axis focus drive 5 moves the objective in the Z direction for focusing. A joystick 6 provides for manual movement (if desired) of the stage in the XYZ direction. A high resolution digital camera 7 acquires images from each well or location on the plate.

There is a camera power supply 8, an automation controller 9, and a central processing unit 10. The PC 11 provides a display 12, and has associated software. The printer 13 provides for printing of a hard copy record.

Like reference symbols in the various drawings indicate like elements.

### DETAILED DESCRIPTION OF THE INVENTION

The invention includes methods of predicting the evolution of in situ lesions in a patient by examining a microtubule organizing center of a cell in a tissue sample. It can also involve methods of predicting the development of cancer in a patient by examining a tissue sample for centrosome abnormalities. In addition, the invention includes methods of predicting the degree of aggressiveness of a cancer in a patient by examining a tissue sample for the degree of severity of centrosome abnormalities. These methods can be employed to predict cancer in any tissue that contains centrosomes (e.g., prostate, breast, or uterine cervix, epithelial, lung, colon, brain, and all other carcinomas). A particular advantage of the invention is that its methods can be carried by human inspection or can be automated. Automation of tissue preparation, examination for centrosome abnormalities, and analysis can enhance the speed, efficiency, and accuracy of the resulting predictions about cancer.

Methods of Analyzing Cells

There are numerous methods that can be used to analyze cells for centrosome defects. Some examples of these methods are provided below.

First, a tissue sample is taken from a patient using standard biopsy techniques. Once taken, the sample can be prepared in a variety of ways. For example, it can be formalin-fixed and paraffin-embedded. Visualizing centrosomes can be enhanced by staining of the tissue (e.g., immunostaining with pericentrin antibodies). Standard histopathologic criteria can be applied to newly prepared hematoxylin- and eosin-stained sections to confirm the presence of carcinoma in situ in the tissue sample (Rosai, J., Akerman's Surgical Pathology, (Mosby, New York), 1996). Once stained, or otherwise prepared for inspection, a microscope (e.g., high-resolution light or electron microscope) or other appropriate device for detecting subcellular structures can be used to detect and view centrosomes.

Reference tissue samples can be used to judge centrosome abnormality. For example, samples of normal, healthy tissue of the same tissue type or origin that contain normal centrosomes can be compared to any tissue samples being assayed for the presence of centrosomal abnormalities.

One method of obtaining a reference tissue sample involves deriving both the tissue sample to be assayed and the reference tissue sample from the same tissue of the same patient. For example, tissue samples can be taken from the same prostate gland of a patient, one sample from a location known to be normal and healthy, and the other from a location to be assayed.

Alternatively, the reference tissue sample can be taken from the same patient at an earlier point in time (analogous to dental records) to be used in the future as a reference. Or, it can be taken from a different patient whose tissue is known to be normal and healthy. Exemplary normal and healthy tissue samples can be preserved and used as references. A reference tissue known to be normal and healthy could be preserved for future comparison. Or, the appearance of a reference tissue known to be normal and healthy could be recorded onto another medium (e.g., an image on paper, a computer image) for visual, or other (e.g., automated or computer), comparison to the tissue to be assayed. Many other methods are possible.

Alternatively, cell lines can be employed. For example, cell lines to be compared (e.g., a normal, healthy cell line and a cell line to assayed) can be grown on glass coverslips in Defined Keratinocyte-SFM media containing 5% fetal bovine serum and antibiotics. After permeabilization of cells in microtubule stabilization buffer containing 0.1% triton-X 100 cells were fixed in cold (−20° C.) methanol and centrosomes immunostained as described in Pihan, G. A., et al. (Cancer Res, 58:3974-85, 1998). Immunofluorescence and FISH can also be employed.

Some examples of centrosomal abnormalities include:

(1) centrosomes with diameters greater than twice the diameter of centrosomes present in normal, healthy samples of the same tissue type or origin,

(2) centrosomes in which the ratio of a centrosome's greatest and smallest diameter exceeds about 1.5-2,

(3) tissues in which there are more than two centrosomes per cell in more than about 5% of the cells examined or yielding a ratio of centrosomes to nuclei of greater than about 2.5,

(4) abnormally shaped centrosomes,

(5) absence of centrosomes,

(6) centrosomes that are organized as multiple small dots in comparison to the organization of normal, healthy centrosomes, and

(7) increased levels (or concentrations) of pericentrin within a cell.

In general, centrosomal abnormalities can include any difference from the centrosomes in samples of normal, healthy tissue of the same tissue type or origin. Differences can be in shape, size, color, orientation, proximity to other cellular or subcellular structures, timing of appearance, movement over time, or any other aspect of appearance or behavior, either at one sampling time or over multiple sampling times.

The frequencies of centrosomal abnormalities in different tissue samples can be compared. For example, the frequency of centrosomal abnormalities in a normal, healthy reference sample can be compared to the corresponding frequency in the tissue being assayed. The increased probability of developing cancer or of developing a more aggressive cancer correlates with the difference in frequency of centrosomal abnormalities between the reference tissue sample and the tissue sample being assaying.

Mitotic spindles can also be examined using similar methods as those used to visualize or detect centrosomal abnormalities. For example, g-tubulin can be used to stain mitotic spindles in archival formalin-fixed paraffin-embedded tissues because it decorates spindle poles while a large fraction of a and b tubulins are cytoplasmic and obscure the spindle microtubule signal.

Automated Centrosome Analysis

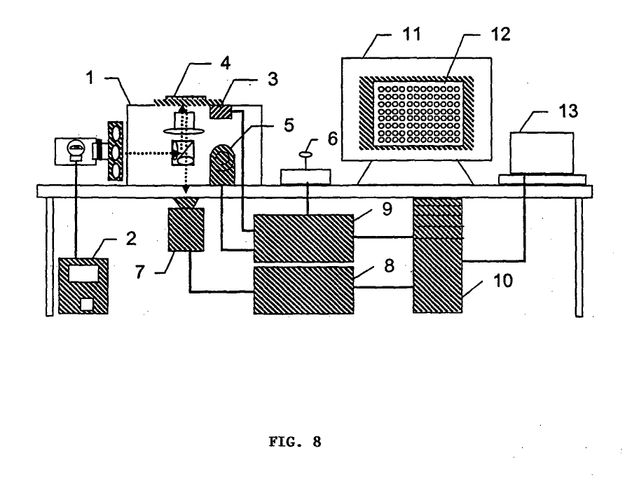

The invention includes automation of any of the above aspects of sampling, examining, or analyzing centrosomal abnormalities. For example, a computer can be programmed to compare images of normal, healthy centrosomes (e.g., shape, color, size, number, orientation, appearance, behavior, etc.) to images of centrosomes from a patient's tissue sample or cell culture. These images can be generated by preparing a the cell tissues or cell cultures in a variety of ways to highlight the centrosomal aspect or aspects of interest so that they can be visualized by a microscope, or other device for visualizing or detecting particular characteristics of centrosomes. Preparation of cell tissues or cell cultures can involve such techniques as staining using a two-color immunofluoresence, two-color immunohistochemistry, or both simultaneously. In addition, automation can allow one to greatly increase the volume of analyses that can be made. For example, one could use punch-embedded paraffin slides to analyze 100 or more tumors per slide for centrosomal abnormalities. FIG. 8 depicts an example of an automated system than can be used to examine and analyze tissue or cell samples for centrosomal abnormalities.

For example, cells from a patient to be examined for centrosomal abnormalities can be cultured using standard cell culture techniques. Then, these cells can be loaded onto an automated system. The system can automatically prepare the cell samples by staining, or some other means of enhancing visualization. Then, the system can examine the samples using a microscope. The microscope can visualize characteristics of interest in the samples, and then transmit information regarding those characteristics to a computer. The computer can then compare characteristics of interest in the cells (e.g., shape, size, color, or number of centrosomes) to reference characteristics (e.g., of normal, healthy cells, or of previously analyzed samples taken from the same patient). The computer can be programmed to decide whether or not the centrosomes in one sample are sufficiently similar to or different from those in a different sample to allow a prediction regarding a cancer, and, if so, to identify a particular prediction.

The invention includes the use of a high magnification, high resolution, three-dimensional acquisition microscope. The microscope can be a microscope capable of taking pictures in a Z-series that can visualize centrosomes in all planes of a cell. The light source can be white light, fluorescence, or multiple wavelength fluorescence.

The invention can use conventional immunohistochemical methods or immunofluorescence methods, using conventional methods for preparing samples for immunohistochemistry or immunofluorescence.

As a practical example, a patient could provide a tissue sample at age 20, which could be examined and analyzed using an automated system, and the resulting centrosomal information stored in her medical records. Then, the patient could provide a second tissue sample at age 25 (and at subsequent intervals), which could be examined and analyzed again, and then compared to results for the original sample. A change in centrosomal characteristics (e.g., a statistically significantly greater ratio of centrosomes to nuclei in the latter sampled tissue compared to the earlier sampled tissue) could result in a prediction that the patient is undergoing early development of cancer in that tissue. The patient could then begin cancer therapy earlier than if she had waited until symptoms of cancer appeared. Her chances for survival might thus be increased.

There are many ways in which the methods of this invention can be automated. These include any of the methods disclosed in WO 00/26408 and in U.S. Pat. Nos. 6,553,135, 6,418,236, 6,372,183, 6,330,349, 6,328,567, 6,317,617, 6,215,892, 6,200,781, 6,190,170, 6,127,133, 6,088,473, 6,048,314, 6,011,862, 5,984,870, 5,812,419, 5,790,690, 5,717,602, 5,656,499, 5,650,122, 5,631,165, 5,620,898, 5,526,258, and 5,509,042, all of which are hereby incorporated by reference in their entirety. Examples of commercially available systems that can be used to automate examination and analysis of centrosomal abnormalities in tissue or cell samples are the Discovery-1™ or Discovery-TMA™ systems (along with MetaMorph®, MetaFluor®, or MetaVue™ systems) from Molecular Devices Corporation.

Centrosome Abnormalities

Chromosomal instability (CIN) is believed to be caused by continuous chromosome missegregation during mitosis and is the most common form of genetic instability in human cancer (Lengauer C., et al., Nature, 396:643-9, 1998). Together with structural chromosome changes, CIN is thought to be important to promote Darwinian genomic evolution characteristics of cancer (Cahill, D. P., et al., Trends Cell Biol, 9:M57-60, 1999). The combined effect of CIN and chromosome breakage and misrepair can explain the progressive loss of tumor suppressor genes and accumulation of extra copies of tumor promoting genes (oncogenes, cell survival genes) characteristic of cancer. In fact, loss of heterozygocity in cancer primarily affects whole chromosomes or large chromosomal domains suggesting that it results from non-disjunction of whole normal or structurally abnormal chromosomes (Thiagalingam, S., et al., Proc Natl Acad Sci USA, 98:2698-702, 2001). CIN is thought to facilitate the inexorable evolution of cancers toward cellular states that support tumor cell growth, dissemination, and resistance to therapy (Lengauer C., et al., Nature, 396:643-9, 1998; Cahill, D. P., et al., Trends Cell Biol, 9:M57-60, 1999; Lengauer, C., et al., Nature, 386:623-7, 1997; Pihan, G. A., et al., Cancer Res, 58:3974-85, 1998; Pihan, G. A., et al., Semin Cancer Biol, 9:289-302, 1999). A common element in the chain of events associated with loss of fidelity in chromosome segregation is centrosome dysfunction (for review, see Pihan, G. A., et al., Semin Cancer Biol, 9:289-302, 1999; Brinkley, B. R., Trends Cell Biol, 11:18-21, 2001; Doxsey, S., Nat Rev Mol Cell Biol, 2:688-98, 2001; Lingle, W. L., et al., Curr Top Dev Biol, 49:313-29, 2000; Marx, J., Science, 292:426-9, 2001; Winey, M., Curr Biol, 9:R449-52, 1999).

Centrosomes are the primary microtubule-organizing centers in animal cells, and they contribute to the organization of microtubule spindles in mitosis and control progression through cytokinesis and entry into S phase (Doxsey, S., Nat Rev Mol Cell Biol, 2:688-98, 2001; Hinchcliffe, E. H., et al., Genes Dev, 15:1167-81, 2001; Khodjakov, A., et al., J Cell Biol, 153:23742, 2001; Piel, M., et al., Science, 291:1550-3, 2001). Centrosome defects have been detected in aggressive carcinomas of multiple origins (Pihan, G. A., et al., Cancer Res, 58:3974-85, 1998; Lingle, W. L., et al., Proc Natl Acad Sci USA, 95:2950-5, 1998). The invention is based, at least in part, on the discovery that centrosome defects in a tissue are strongly correlated to whether or not that the tissue will develop cancer, the evolution of such a cancer, and the resulting severity of that cancer.

The established role of centrosomes in organizing mitotic spindles suggested a model in which tumor cells with multiple centrosomes organize multipolar spindles that in turn missegregate chromosomes and contribute to genetic instability. This phenomenon can occur in diploid cells or in cells that previously failed in cell division to create polyploid cells with excess centrosomes (Meraldi, P., et al., Embo J, 21:483-92, 2002). Despite the occurrence of centrosome defects in human cancers, and their important role in the assembly of mitotic spindles and chromosome segregation, a role for centrosomes in the earliest steps of human tumor development has not elsewhere been established. The invention is based, at least in part, on the discovery that centrosome defects and genetic instability occur in some low grade prostate tumors and are present prior to development of aggressive tumors. However, it appears that centrosome defects have not previously been linked to the earliest stages of human cancer where they would have the highest potential to contribute to the early stages of the disease, and possibly serve as prognostic markers for tumor development and therapeutic targets for treatment.

Pre-invasive cancer lesions in humans known as carcinoma in situ provide a unique opportunity to directly examine this issue in some detail. This invention is based, at least in part, on the recognition that centrosome defects occur in carcinomas in situ from multiple tissue sources and co-segregate with other tumor-like features associated with centrosome dysfunction, such as spindle abnormalities, cytologic changes, and chromosomal instability.

Centrosome Defects and Precancerous Lesions

Experimental results upon which this invention is based, at least in part, demonstrate that centrosome defects play a critical role in carcinogenesis. Centrosome defects occur frequently in advanced forms of some of the most common human cancers, and contribute to genetic instability by impairing the fidelity of chromosome segregation during mitosis (Lengauer C., et al., Nature, 396:643-9, 1998; Cahill, D. P., et al., Trends Cell Biol, 9:M57-60, 1999; Brinkley, B. R., Trends Cell Biol, 11:18-21, 2001; Doxsey, S., Nat Rev Mol Cell Biol, 2:688-98, 2001; Marx, J., Science, 292:426-9, 2001; Lingle, W. L., et al., Proc Natl Acad Sci USA, 95:2950-5, 1998). Carcinoma in situ is the immediate precursor of invasive epithelial cancers and it shares some, but not all, genotypic and phenotypic characteristic of invasive cancer (Bostwik, D. G., Semin Urol Oncol, 17:187-98, 1999; Shultz, L. B., et al., Curr Opin Oncol, 11:429-34, 1999; Wolf, J. K., et al., Cancer Invest, 19:621-9, 2001). The experimental results disclosed herein show that centrosome defects are present at the earliest morphologically recognizable stages of tumor development in some of the most common human cancers. They provide a mechanistic explanation for the commonly observed CIN and aneuploidy observed in most lesions found in experimental models of carcinogenesis and human carcinoma in situ (Bulten, J., et al., Am J Pathol, 152:495-503, 1998; Levine, D. S., et al., Proc Natl Acad Sci USA, 88:6427-31, 1991; Li, R., et al., Proc Natl Acad Sci USA, 94:14506-11, 1997; Wang, X. W., et al., Proc Natl Acad Sci USA, 96:3706-11, 1999; Weinberg, D. S., et al., Arch Pathol Lab Med. 117:1132-7, 1993). These data demonstrate the presence of centrosome defects in the generation of genetic instability during the early stages of the tumorigenic process.

Furthermore, centrosome defects correlate with the histologic/cytologic grade of the in situ lesion, and the centrosome has a role in the induction of the morphologic phenotype characteristic of carcinoma in situ. Centrosomes have been shown to play a role in cell polarity, shape, and motility, all of which are perturbed in in situ cancers. Moreover, the presence of mitotic spindle defects in many carcinoma in situ of the uterine cervix (CIC, or carcinoma in situ of the cervix) and carcinoma in situ of the female breast (DCIS, or ductal carcinoma in situ) lesions, and the co-segregation of centrosome abnormalities with CIN in these lesions, show that centrosome defects have an important functional impact in in situ carcinoma.

The experimental results herein demonstrate a role for centrosome defects in the development of aggressive tumors, rather than those that remain benign. For example, there is a high prevalence of centrosome abnormalities in lesions with a high rate of progression to high-grade cancer (DCIS (ductal carcinoma in situ) and CIC (carcinoma in situ of the cervix)), and a low prevalence of centrosome defects in lesions associated with progression to low grade invasive cancers, such as prostate intraepithelial neoplasia (PIN). It has been shown that most invasive cancers of the breast and uterine cervix are aggressive high-grade cancers. Because DCIS and CIC are usually indistinguishable cytologically from aggressive cancers it is believed that they give rise to these aggressive cancers. In contrast, cancers of the prostate are usually low-grade cancers consistent with the low-grade appearance of most PIN lesions. These results support the centrosome-mediated model of tumorigenesis where centrosome defects induce dramatic and persistent changes in chromosome number, thereby shuffling the genome and allowing selection of the most aggressive phenotypes such as those seen in invasive cancers.

The invention is based, at least in part, on the discovery that the presence of centrosome abnormalities in cells at the earliest stages of disease allows prediction of the evolution of in situ lesions into high-grade invasive cancers. This discovery is of particular interest for the management of prostate cancer since the majority of these tumors are biologically low grade. Currently, these cancers are often treated by prostatectomy because there is no effective prognostic indicator of aggressive disease. Since centrosome abnormalities predict the development of high grade cancer, such prediction can provide a sorely needed surrogate marker for high grade cancer. Centrosome defects correlate with aggressive disease, as can be shown by examining PIN lesions from patients who subsequently progressed to invasive cancer. Centrosome defects in early (precancerous) lesions are worse in lesions that subsequently progress to worse, or more aggressive, tumors.

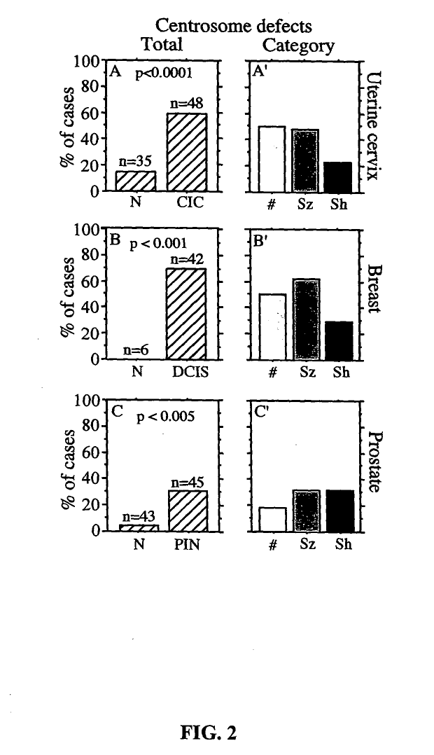

An interesting observation was the presence of low, yet measurable, levels of centrosome defects in morphologically normal epithelium adjacent to CIC lesions (FIG. 2A). This may be due to the presence of human papillomavirus infection. It is well established that papillomavirus is the cause of nearly all carcinomas of the cervix, and is present in all precursor lesions (Munger, K., Front Biosci, 7:d641-9, 2002). Moreover, it has recently been demonstrated that papillomavirus can rapidly induce centrosome abnormalities in squamous epithelial cells (Duensing, S., et al., Biochim BiophysActa, 2:M81-8, 2001).

Another important discovery is the functional impact of abnormal centrosomes in in situ carcinomas. It has been demonstrated in experimental systems and cell lines (Brinkley, B. R., Trends Cell Biol, 11:18-21, 2001; Ring, D., et al., J Cell Biol, 94:549-56, 1982) that multipolar spindles formed by supernumerary centrosomes may coalesce to form bipolar spindles, mitigating the functional consequences of centrosome defects on chromosome segregation. Whether coalescence occurs in in situ cancers is not know. However, even if it does, it is not sufficient to completely suppress the effect of supernumerary centrosomes on spindle multipolarity.

Whether centrosome defects are cause or consequence of the in situ carcinoma phenotype, centrosomal abnormalities can be predictive of the development of cancer. Thus, identification of centrosomal abnormalities can be important for predictive testing and effective cancer-specific therapeutic interventions. There are many ways in which centrosome defects can arise. These include changes in proteins involved in cell cycle control, in centrosome structure or function, and in DNA repair. For instance, mutation or elimination of p53 (Fukasawa, K., et al., Science, 271:1744-7, 1996; Tarapore, P., et al., Oncogene, 20:3173-84, 2001; Wang, X. J., et al., Oncogene, 17:35-45, 1998), or p53 downstream effectors or regulators, such as Mdm2 (Carroll, P. E., et al., Oncogene, 18:1935-44, 1999), p21Waf/Cip1 (Fukasawa, K., et al., Science, 271:1744-7, 1996; Carroll, P. E., et al., Oncogene, 18:1935-44, 1999; Mantel, C., et al., Blood, 93:1390-8, 1999), or GADD45 (Wang, X. W., et al., Proc Natl Acad Sci USA, 96:3706-11, 1999; Hollander, M. C., et al., Nat Genet, 23:176-84, 1999), induce centrosome abnormalities. Abrogation of postmitotic p53-dependent checkpoints may be critical in allowing tetraploid cells with supernumerary centrosomes to continue to cycle (Andreassen, P. R., et al., Mol Biol Cell, 12:1315-28, 2001; Khan, S. H., et al., Cancer Res, 58:396-401, 1998; Lanni, J. S., et al., Mol Cell Biol, 18:1055-64, 1998). Similarly, alteration in the levels of centrosome-associated proteins such as pericentrin (Pihan et al., Cancer Res, 61:2212-9, 2001; Purohit, A., et al., J Cell Biol, 147:481-92, 1999), g-tubulin (Shu, H. B., et al., J Cell Biol, 130:1137-47, 1995), aurora (Meraldi, P., et al., Embo J. 21:483-92, 2002; Bischoff, J. R., et al., Embo J. 17:3052-65, 1998; Zhou, H., et al., Nat Genet, 20:189-93, 1998), polo (Conn et al., Cancer Res., 60:6826-31), TACC (Raff, J. W., et al., Cell, 57:611-9, 1989), and RanBP (Wiese, C., et al., Science, 291:653-6, 2001) lead to abnormal centrosomes. Moreover, mutation or functional abrogation of proteins involved in DNA repair such as Xrcc3 (Griffin, C. S., et al., Nat Cell Biol, 2:757-61, 2000), Xrcc2 (Griffin, C. S., et al., Nat Cell Biol, 2:757-61, 2000), BRCA1 (Bertwistle, D., et al., Breast Cancer Res, 1:41-7, 1999; Xu, X., et al., Mol Cell, 3:389-95, 1999), BRCA2 (Kraakman-van der Zwet, M., et al., Mol Cell Biol, 22:669-79, 2002; Tutt, A., et al., Curr Biol, 9:1107-10, 1999), Mre11 (Yamaguchi-Iwai, Y, et al., Embo J. 18:6619-29, 1999), or DNA polymerase beta (Bergoglio, V., et al., Cancer Res, 62:3511-4, 2002), or genome damage signaling proteins such as ATR (Smith, L., et al., Nat Genet, 19:39-46, 1998) can also lead to centrosome abnormalities. Lastly, centrosome abnormalities can also arise by mutation of the adenomatous polyposis coli gene (APC) whose product interacts with microtubules (Foddle, R., et al., Nat Cell Biol, 3:433-8, 2001), by cytokinesis failure (Doxsey, S., Nat Genet, 20:104-6, 1998), and by ectopic assembly of centrosome components into acentriolar microtubule organizing centers (Doxsey, S., Nat Rev Mol Cell Biol, 2:688-98, 2001; Pihan, G. A., et al., Cancer Res, 61:2212-9, 2001; Purohit, A., et al., J Cell Biol, 147:481-92, 1999).

### EXAMPLES

The invention is further described in the following examples, which do not limit the scope of the invention described in the claims. The general experimental procedures are described first.

Experimental Procedures

Immunohistochemical Staining and Analysis

Formalin-fixed paraffin-embedded tissue from carcinoma in situ of the uterine cervix, female breast, and male prostate was selected from the files of the Pathology Department at UMass Memorial Health Care. Samples were immunostained with pericentrin antibodies as described (Pihan, G. A., et al., Cancer Res, 58:3974-85, 1998; Pihan et al., Cancer Res, 61:2212-9, 2001; Purohit, A., J Cell Biol, 147:481-92, 1999). Standard histopathologic criteria was applied to newly prepared hematoxylin and eosin stained sections to confirm the presence of carcinoma in situ in the specimen (Rosai, J., Akennan's Surgical Pathology, (Mosby, New York), 1996). Centrosomes were considered abnormal if they had a diameter greater than twice the diameter of centrosomes present in normal epithelium within the same section, if the ratio of a centrosome's greatest and smallest diameter exceeded 2, or if there were more than two centrosomes in more than 5% of the cells examined (Pihan et al., Cancer Res., 61:2212-2219, 2001). g-tubulin was chosen to stain mitotic spindles in archival formalin fixed paraffin embedded tissues because it decorates spindle poles while a large fraction of a and b tubulins are cytoplasmic and obscure the spindle microtubule signal. Multipolar mitoses, an obvious consequence of supernumerary centrosomes, are common in carcinoma cell lines with abnormal centrosomes as we and others have previously shown (Pihan et al., Cancer Res., 58:3974-85, 1998; Sato et al., Clin. Cancer Res., 5:963-70, 1999; Lingel et al., Am. J. Pathol., 155:1941-51, 1999; Saunders et al., PNAS, 97:303-8, 2000).

Chromosomal Instability Analysis

Tissue sections parallel to those used for pericentrin immunohistochemistry were used to stain for the centromeres of chromosome 1 and 8 (Pihan et al., Cancer Res., 58:3974-85, 1998). Briefly, after de-paraffinization, sections were co-denatured with biotinylated centromeric probes specific for chromosomes 1 or 8 and hybridized overnight at 37° C. in a Hybrite oven (Vysis, Chicago, Ill.) in the hybridization buffer recommended by the probe manufacturer. After appropriate stringency washes sections were placed on the automatic immunostainer and an ABC/DAB protocol similar to the one used above for immunohistochemistry was used to reveal the hybridized probe. Nuclei were lightly counterstained with hematoxylin. For quantitative analysis, the number of hybridization signals in 100 to 200 nuclei from in situ carcinoma and morphologically normal adjacent epithelium was recorded (Pihan et al., Cancer Res., 58:3974-85, 1998). Using these probes it has been shown that normal diploid tissue has 10-15% cells with more than 3 signals per nucleus (Pihan et al., Cancer Res., 58:3974-85, 1998; Bulten et al., Am. J. Pathol., 152:495-503, 1998). In tissue sections some nuclei are truncated leading to artificially increased numbers of diploid cells with apparently less than two signals per nuclei. For this reason, computed signal gains (greater than two) were computed, and not apparent losses. Due to this limitation, no attempt was made to obtain an absolute measure of chromosome instability in sections, as it can be done on cell lines (Lengauer et al., Nature, 386:623-7, 1997; Pihan et al., Cancer Res., 58:3974-85, 1998). Rather, tumors with likely aneuploidy/CIN were defined as those in which the fraction of nuclei with more than two signals exceeded 20% (Bulten et al., Am. J. Pathol., 152:495-503, 1998), and used this measurement as an index of chromosome instability/aneuploidy.

Analysis of Cell Lines Derived from PIN or Normal Prostate Epithelium

During attempts to establish isogenic pairs of neoplastic and normal epithelial cell lines from patients with prostate cancer at NCI, one pair of normal and high grade PIN cell lines was derived from the same patient (Bright et al., Cancer Res., 57:995-1002, 1997). Pathologic examination of the donor prostate showed only normal glands and extensive high grade PIN, but no invasive carcinoma. To study centrosomes, cell lines were grown on glass coverslips in Defined Keratinocyte-SFM media containing 5% fetal bovine serum and antibiotics. After permeabilization of cells in microtubule stabilization buffer containing 0.1% triton-X 100 cells were fixed in cold (−20° C.) methanol and centrosomes immunostained as described (Pihan et al., Cancer Res., 58:3974-85, 1998). In situ hybridization with probes to chromosomes 1 and 8 were carried out as previously described (Pihan et al., Cancer Res., 58:3974-85, 1998). Immunofluorescence and FISH were carried out in four different experiments and results averaged.

### Example 1

### Centrosome Defects Occur in a Significant Number of Pre-Invasive Cancerous Lesions

Carcinoma in situ of the uterine cervix (CIC), the female breast (DCIS), and the male prostate (PIN) was studied. These lesions are precursors of the most common human cancers. Moreover, breast and prostate cancers are the second leading cause of cancer death in women and men, respectively.

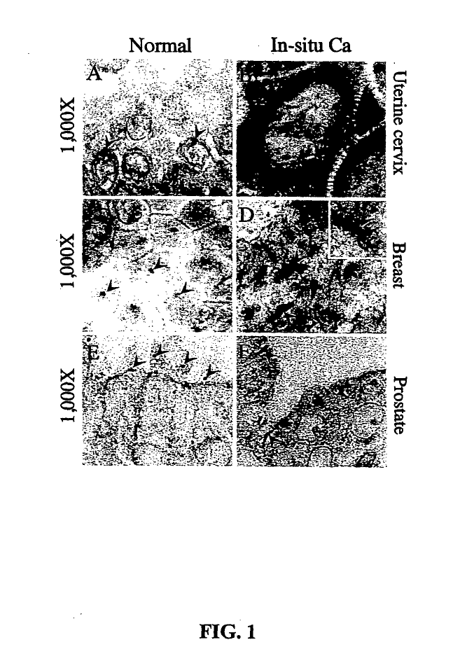

Using antibodies to the centrosome protein pericentrin (Doxsey et al., Cell, 76:639-50, 1994), we examined microtubule organizing centers in sections of tumor and nontumor tissues as described (Pihan et al., Cancer Res., 58:3974-85, 1998; Pihan et al., Cancer Res., 61:2212-2219, 2001). Several distinct centrosome abnormalities were detected in these lesions, including supernumerary centrosomes (FIG. 1B arrowheads), abnormally-shaped centrosomes, such as elongated or cork-screw forms (FIGS. 1D and F) and centrosomes of larger diameter than those in normal epithelium within the same tissue section (FIGS. 1B and D). Also observed were cells that apparently lacked centrosomes, or whose centrosomes were organized as multiple small dots. Because this phenotype could partly be a consequence of cell truncation during tissue sectioning, these were not scored as defects even though a similar phenotype was observed in tumor cell lines. Quantification of centrosome defects in all precancerous lesions demonstrated that 36-72% had abnormal centrosomes (FIGS. 2A-C), while nontumor cells had undetectable or low levels of defects (FIGS. 2A-C). Centrosome defects were more prevalent in DCIS and CIC lesions than in PIN lesions. Differences in centrosome abnormalities between DCIS and CIC, on one hand, and PIN, on the other, are consistent with differences in histological, cytological, and genetic features of these lesions. DCIS and CIC show a high degree of nuclear atypia, cytologic disarray, loss of cell polarity, and genetic instability. In fact, on cytologic features alone, they are often indistinguishable from invasive breast and cervical cancers (Crum et al., J. Cell. Biochem. Suppl., 23:71-9, 1995; O'Connell et al., Breast Cancer Res. Treat., 32:5-12, 1994). This is in contrast to PIN lesions that show preservation of cell polarity, and glandular architecture, and can only be distinguished from normal glands by rather subtle changes in nuclear and nucleolar features.

In summary, it was demonstrated that centrosome abnormalities occur in pre-invasive lesions, and that they are more common in CIC and DCIS than in PIN lesions. Similar results were obtained using g-tubulin antibodies in interphase cells, although fewer defects were observed than with pericentrin antibodies.

### Example 2

### The Incidence of Centrosome Defects Increases with Higher Histologic Grade of In Situ Carcinomas

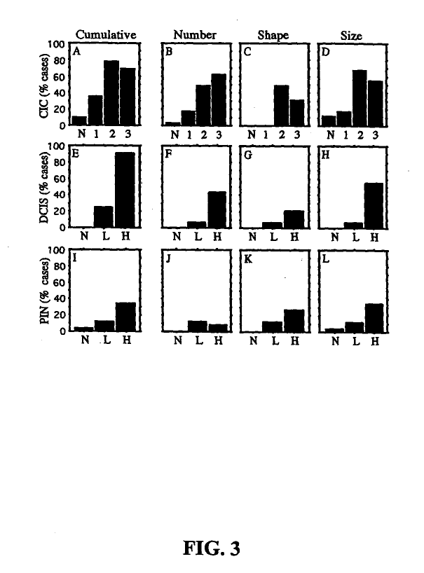

In situ carcinomas of different histologic/cytologic grade differ in their associated risk of progression to invasive carcinoma. A 2-4-fold increase in the incidence of centrosome defects with increasing histologic/cytologic grade in all three precancerous lesions was observed (FIG. 3). Most DCIS lesions exhibited centrosome defects (FIG. 3E), while only 36% of high-grade PIN lesions had this phenotype (FIG. 31). The surprisingly high incidence of centrosome defects in DCIS is consistent with the cytologic similarity between DCIS and invasive breast cancer (Pihan et al., Cancer Res., 58:3974-85, 1998). CIC lesions of histologic grade 2 and 3 (collectively “high grade” lesions) showed a high incidence of centrosome defects, nearly as high as that seen in DCIS lesions (FIG. 3A). Centrosome abnormalities in all three types of lesions was greater in those lesions associated with a higher propensity to evolve into invasive carcinoma. This trend demonstrates an important role for centrosomes in generating the cytologic and genetic changes that occur during tumor progression.

### Example 3

### Mitotic Spindle Abnormalities are Frequent in Carcinoma In Situ

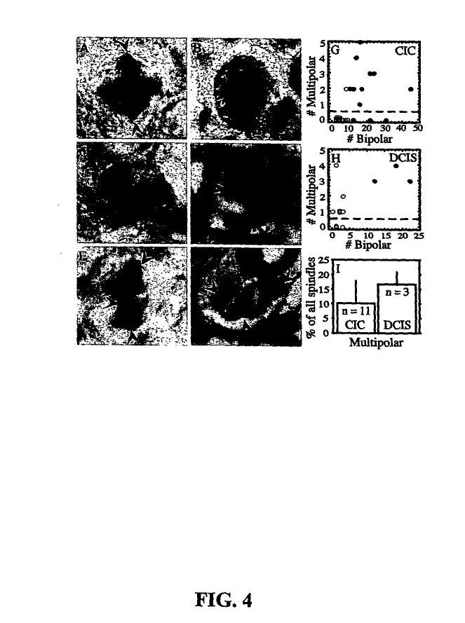

One expected consequence of supernumerary centrosomes in mitotic cells is the development of multipolar mitotic spindles (Pihan et al., Cancer Res., 58:3974-85, 1998; Purohit et al., J. Cell. Biol., 147:481-92, 1999). To identify abnormal spindles, sections were stained with g-tubulin, which provided the best marker for spindle poles in this immunohistochemical procedure (see Experimental procedures). Although the total number of mitotic figures was generally low, mitotic spindles were found in 74% (29/39) of CIC lesions, 35% (12/34) of DCIS lesions, and in none of the PIN lesions (0/42) and nontumor cells. The low incidence of spindles in PIN lesions is likely the result of delayed fixation and the relatively slow growth of prostate tumor cells compared with the other in situ lesions (DCIS, CIC). Of the tumors with spindles, 75% (9/12) of DCIS and 34% (10/29) of CIC had at least one abnormal spindle (FIGS. 4H and G). Defective spindles included multipolar spindles (3 or more poles, FIGS. 4B, D, and F), multiple bipolar spindles in single cells (FIG. 4E), and asymmetric bipolar and multipolar spindles (FIGS. 4D and F).

To get a measure of the extent of this phenotype in in situ lesions, and to avoid the inherent bias introduced in the data by low spindle counts, abnormal spindles in cells with 10 or more spindles were counted. The average number of multipolar spindles in cases so selected was 10.1+/−7.8 and 16.6+/−4.1, respectively (FIG. 41). Monopolar spindles were also detected, but they could not be authenticated due to the compounding effect of truncation artifacts induced by tissue sectioning. Mitotic figures were infrequently observed in normal epithelium adjacent to lesions. This is most likely due to the low mitotic rate of these tissues, but in all cases they appeared structurally normal (symmetric, bipolar, n=4). Because of the low incidence of spindles in nontumor tissues, and to control for the nonspecific effects of the immunohistochemical procedure on mitotic cells, results from in situ carcinomas were compared with those of a highly proliferative epithelium. In biopsies from patients with celiac sprue, a form of malabsortion, the small intestinal epithelium has increased mitotic activity due to increased rates of mucosal regeneration. In these biopsies, abnormal mitoses (n=45) were never observed, indicating that the observations in in situ carcinomas are not an artifact of staining in archival tissue biopsies.

### Example 4

### Centrosome Defects Correlate with CIN in Precancerous Lesions

Both chromosome instability (Lengauer C., et al., Nature, 396:643-9, 1998; Pihan et al., Cancer Res., 58:3974-85, 1998; Lingle et al., PNAS, 95:2950-5, 1998) and centrosome defects are common features of epithelial cancers (Marx, Science, 292:426-9, 2001; Lingle et al., PNAS, 95:2950-5, 1998; Pihan et al., Cancer Res., 61:2212-9, 2001; Lingle et al., Am. J. Pathol., 155:1941-51, 1999). To determine whether a correlation exists between centrosome defects and CIN in carcinoma in situ, consecutive serial tissue sections were examined for these anomalies (for methods, see Pihan et al., Cancer Res., 58:3974-85, 1998; Ghadami et al., Genes Chromosomes Cancer, 27:183-90, 2000; Pihan et al., Cancer Res., 61:2212-9, 2001; Bright et al., Cancer Res., 57:995-1002, 1997).

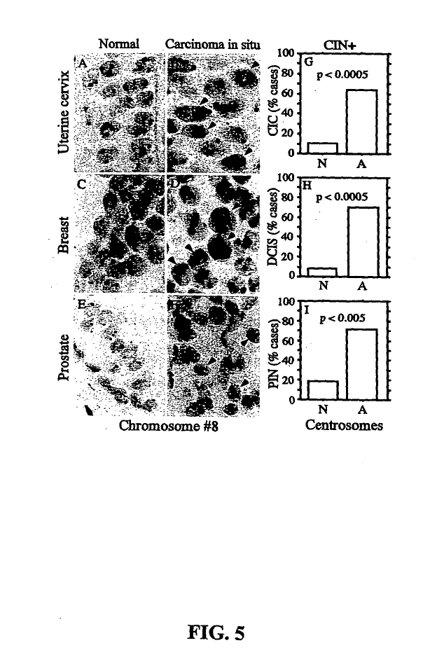

While CIN was observed in many in situ lesions, it was never seen in normal epithelium in the same tissue section (FIGS. 5A, C, and E). Moreover, in all three in situ carcinomas there was a statistically significant non-random association (Fisher exact test p<0.005) between centrosome defects and CIN (FIGS. 5G-I). In fact, most lesions with centrosome defects showed CIN (63-71%, FIG. 5). Conversely, the fraction of cases that lacked centrosome defects, lacked CIN (81-95%, FIG. 5). This correlation between centrosome defects and CIN was significant despite the vastly different degrees of centrosome defects between DCIS, CIC, and PIN (FIG. 2). Interestingly, there were more lesions that had centrosome defects and no CIN (˜30%) than lesions with CIN and no centrosome defects (˜10-20%), showing that centrosome defects precede CIN in the progression of the tumor-like phenotype in precancerous lesions (Pihan et al., Cancer Res., 58:3974-85, 1998; Doxsey, Nat. Rev. Mol. Cell. Biol., 2:688-98).

Thus, centrosome abnormalities can be used to predict CIN and the development and progression of a cancer.

### Example 5

### Centrosome Abnormalities and CIN in Cell Lines Derived from PIN and Normal Tissues

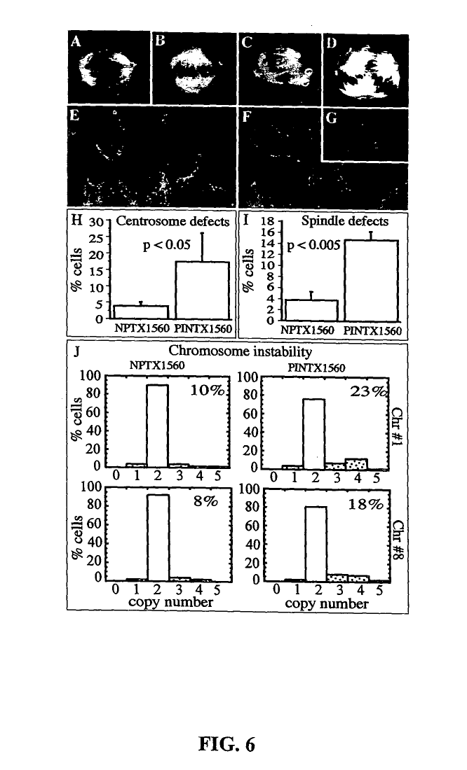

One of the only known in situ carcinoma cell lines available (Bright et al., Cancer Res., 57:995-1002, 1997) was investigated for centrosome defects and CIN. Cell lines provide a better quantitative measure of these features and can ultimately be used to examine the molecular mechanism responsible for centrosome abnormalities. A line derived from a high-grade PIN lesion (1560PINTX) and a control line derived from normal prostate epithelium (1560NPTX) both originated from the same surgically-excised prostate gland (Bright et al., Cancer Res., 57:995-1002, 1997). Immunofluorescence analysis using pericentrin antibodies to detect centrosome defects revealed a significantly higher incidence of centrosome abnormalities in PIN cells than in normal cells (˜4-fold higher, FIG. 6H). As in tumors, the incidence of multipolar spindles paralleled the incidence of centrosome defects, being higher in PIN cells than in normal cells (FIG. 6I). The level of CIN was also consistently higher in PIN-derived cells compared with controls (FIG. 6J).

Thus, centrosome abnormalities can be used to predict CIN and the development and progression of a cancer (e.g., PIN cells).

### Example 6

### Diagnosis of Prostate Cancer

The etiology of prostate carcinoma is unknown. Understanding the fundamental cellular mechanisms involved in disease onset and progression is essential for designing methods for the detection and treatment of this major form of human cancer. This invention allows the development of early and effective prognostic methods for aggressive disease and production of novel therapies based on the identification of new targets for prostate cancer.

Prostate tumor virulence correlates with aberrant cytoarchitecture (Gleason grades 4, 5) and high grade tumors exhibit genetic instability. However, little is known about the molecular and biologic basis of these aberrant cellular features. Centrosomes and associated microtubules play a critical role in mitosis by coordinating spindle assembly and cytokinesis with chromosome segregation and in interphase by regulating cell polarity and shape. All these processes are disrupted in prostate carcinoma. Several significant observations demonstrate that centrosomes contribute to all known cellular and genetic changes in prostate cancer. Centrosome defects are present in pre-invasive lesions and become more severe during tumor progression, paralleling changes in Gleason grade and genetic instability. Overexpression of the centrosome protein pericentrin produces features indistinguishable from prostate tumor cells and induces or exacerbates prostate cell transformation in vitro. The novel discovery of centrosome defects and elevated pericentrin levels in prostate carcinoma and pre-invasive lesions shows a previously unexplored mechanism for generating the cellular and genetic changes that occur during prostate cancer progression. The observation that pericentrin interacts with several kinases (PKA, PKC, and others) that are themselves implicated in cancer led to the discovery that the oncogenic potential of pericentrin results from loss of pericentrin's interaction with these kinases.

The majority of patients diagnosed with prostate cancer have clinically indolent tumors, while a minority develops more aggressive, often fatal cancer. An effective prognostic test could eliminate the unnecessary treatment of patients with indolent disease, target patients with aggressive disease for early intervention and potentially increased survival, and facilitate better targeting and refinement of therapies. The development of such a test has become ever more critical due to the dramatic increase in the population at risk for this age-related cancer (aging Baby Boom generation), and the increased number of individuals diagnosed with prostate cancer through more sensitive measures of prostate specific antigen (PSA). We have determined that centrosomes were abnormal in nearly all aggressive tumors, but only in a fraction of precancerous (PIN) lesions. Centrosome defects in PIN lesions can predict progression to clinically aggressive tumors examined after prostatectomy or death. This approach can be used to develop clinical assays to test for defects in needle biopsies as well as for changes in molecular components of centrosomes in patient sera.

Prostate carcinoma is the most common gender-specific cancer in the United States, accounting for nearly one third of all cancers affecting American men. The lifetime risk of developing invasive prostate carcinoma in the United States stands at ˜20% (37-40), while that of octogenarians, based on histopathologic examination of the prostate at autopsy, approaches 80%. Despite such an alarmingly high incidence, the lifetime risk of dying from prostate carcinoma is much lower, currently estimated to be around 3.6% (1/28, Surveillance Epidemiology, & End Results Website at NCI, 2,001). The trend toward higher incidence and lower mortality will increase in the next few decades due to the combination of two factors: 1) the aging of the Baby Boom generation, which will result in an increase in the population at risk for this age-dependent cancer, and 2) the clinical implementation of ever more sensitive assays for prostate specific antigen (PSA), which are able to detect increasingly smaller cancer burdens long before the development of clinical symptoms. However, it is currently impossible to predict tumor behavior by non-invasive means, so radical treatment is suggested for essentially all patients with disease, highlighting the critical need to develop a non-invasive test to distinguish clinically indolent (low grade) carcinoma from potentially fatal disease (high grade). Such a test could spare the majority of patients with indolent prostate cancer from unneeded prostatectomy, thus accruing significant cost savings in health care and avoiding much therapy-related morbidity. This test would also enable caretakers to focus therapy on the more homogeneous group of patients with aggressive disease, where the efficacy of newer therapies could be assessed more quickly.

Currently, one of the best predictors of prostate cancer progression is the Gleason score. Because the Gleason score is well known to one of ordinary skill in the art, its details are not provided here. This score is a measure of progressively aberrant cytoarchitectural features (cytologic anaplasia) and glandular de-differentiation, recorded as Gleason grades. Recent results indicate that the proportion of the tumor with the highest Gleason grades (4, 5) appears to have greater predictive power than the Gleason score itself. The intimate relationship between features of high Gleason grades (progressive glandular de-differentiation, cytologic anaplasia) and genetic instability (aneuploidy) suggests that these tumor-associated features may be mechanistically linked. Thus, defects in molecular components and subcellular structures that control cell and tissue architecture and genetic fidelity are likely to contribute to tumor progression and dictate the clinical behavior of tumors, and, thus, to predict aggressive cancer. We have searched for the biological factors that contribute to the constellation of features found in high Gleason grade prostate carcinoma in order to exploit these unexplored factors for disease diagnosis and therapy.

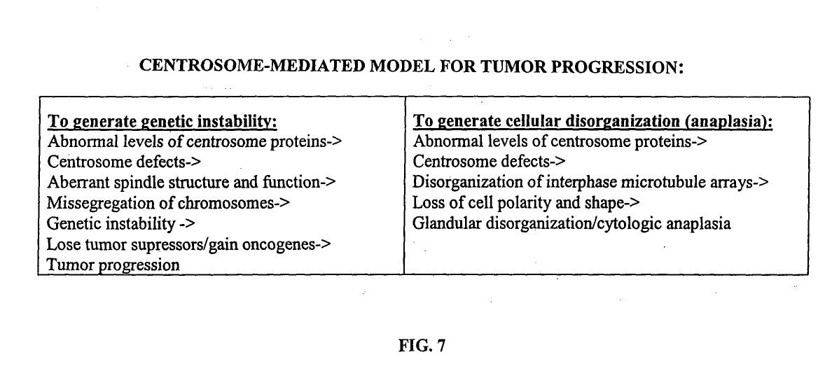

All features of high grade prostate carcinoma result from a previously overlooked phenomenon, namely, defects in centrosome structure and function. Loss of glandular differentiation, cell shape and polarity, and the development of genetic instability could all be caused by centrosome dysfunction. Centrosomes are tiny cellular organelles that nucleate microtubule growth in interphase and mitosis and organize the mitotic spindle to mediate chromosome segregation into daughter cells. As organizers of microtubules, centrosomes also play an important role in many microtubule-mediated processes, such as establishing cell shape and cell polarity, processes essential for epithelial gland organization. Centrosomes also coordinate numerous intracellular activities, in part by providing docking sites for regulatory molecules such as those that control cell cycle progression, centrosome and spindle function, and cell cycle checkpoints. The invention is based, at least in part, on the elucidation of a centrosome-mediated model for prostate tumor progression (FIG. 7).

Centrosomes are defective in the majority of aggressive prostate carcinomas and centrosome defects increase with increasing Gleason grade. Centrosome defects in prostate tumors correlate with genetic instability, loss of normal cellular architecture, and glandular dedifferentiation, demonstrating a strict relationship between defective centrosomes and these tumor-associated features. We discovered that a fraction (˜20%) of precursor lesions to prostate carcinoma (prostate intraepithelial neoplasia, PIN) have abnormal centrosomes. This exciting observation has important implications for prostate cancer etiology and prognosis. The presence of dysfunctional centrosomes early in the tumorigenic process demonstrated that they contribute to genetic instability and cytologic anaplasia that occur later in the disease, and that they can predict development of high grade carcinomas. Data also shows that a similar fraction of PIN lesions exhibit aneuploidy, an indicator of aggressive disease.

The most compelling experimental evidence for our centrosome-based model for prostate cancer progression is the remarkable observation that genetic instability and cellular changes characteristic of advanced Gleason grades can be induced in normal cells and exacerbated in tumor cells by overexpressing the centrosome protein pericentrin. Pericentrin is essential for centrosome and spindle organization and function. Artificial elevation of pericentrin levels induces genetic instability, cytologic anaplasia, centrosome defects, microtubule disorganization, and spindle dysfunction in human, mouse, and monkey cells and normal prostate cells, and exacerbates these features in prostate tumor cells. These cells exhibit other tumor-like features, such as accelerated growth in vitro and aberrant mitotic checkpoint control. Moreover, pericentrin levels are elevated in tumors and in the subset of PIN lesions with centrosome defects. Thus, pericentrin is strongly involved in tumor progression.

Pericentrin interacts with PKA, PKC, and others. The central role of pericentrin in tumor-related functions is mediated through interactions with several essential cellular components. Among these are proteins involved in the nucleation of centrosomal microtubules (e.g., g tubulin) and assembly of pericentrin onto centrosomes cytoplasmic dynein. Pericentrin also interacts with protein kinases that are themselves involved in cancer, namely PKA, PKC, and others. The tumor-like features of pericentrin lie in domains that bind PKA, PKC, and others. All three kinases bind pericentrin (PKA, PKC, and others). Expression of the PKC binding domain of pericentrin uncouples the pericentrin-PKC interaction in the cell and induces aneuploidy (binucleate cells) through cytokinesis failure. In a converse experiment, expression of the pericentrin-binding domain of PKC induces cytokinesis failure and aneuploid cells. The phenotype is specific for PKC bII as 7 other isoforms have little effect on aneuploidy. Disruption of the pericentrin-PKA interaction by similar methods produces spindle defects and binucleate cells. Importantly, expression of a pericentrin mutant lacking the PKA binding domain produces a less severe phenotype than the full-length protein, showing that PKA binding to pericentrin contributes to pericentrin-induced aneuploidy. The pericentrin-bound fraction of all three kinases act independently or cooperatively to control genetic fidelity, and disruption of any of these interactions (e.g., by pericentrin overexpression) induces aneuploidy.

Through its interaction with molecules that are individually essential for spindle function, cytokinesis and chromosome segregation, pericentrin can be viewed as a hub of activities involved in maintaining genetic stability. It is easy to imagine how elevated pericentrin levels disrupt these activities and induce features of aggressive prostate cancer. For example, spindle defects or cytokinesis failure lead to genetic instability, while breakdown in microtubule arrays could cause changes in cell polarity and shape leading to glandular disorganization. Our pericentrin- and centrosome-mediated model of prostate tumor progression explains all forms of genetic instability both in vivo and in vitro, including chromosomal instability, multiple-DNA-content stemlines, near diploid cancer, as well as hypo- and hypertetraploid tumors.

A novel centrosome protein called centriolin is homologous to two different oncogenes. A domain at the amino terminal region is homologous to oncoprotein 18 or stathmin, while domains in the central region and C-terminus are homologous to transforming acid coiled coil, or TACC, proteins. In studies designed to elucidate centriolin function, we discovered that alteration of protein levels is sufficient to drive cells out of the cell cycle. This was accomplished by reducing cellular levels of centriolin using small interfering RNAs (siRNA/RNAi) or by overexpression of a domain at the N-terminus of the protein. The ability to drive cells out of cycle provides a more powerful method for blocking cell proliferation than arresting cells within the cycle. Moreover, driving cells out of cycle suggests that they may enter a unique senescent state that may ultimately lead induce differentiation. Expression of the amino terminal domain of centriolin can eliminate prostate tumor cells in men with prostate cancer (including late stage cancers) by forcing cell cycle exit, inducing differentiation, and returning cells to normal function. Therapy can be based on imposing a G₀-like state on prostate or any other tumor cells.

Prostate carcinoma is unique among solid tumors including breast, lung, and colon in that there is a relatively wide spectrum of cytologic, biologic, and genetic features ranging from the relatively normal in indolent, low grade, carcinomas to the extensively abnormal in high grade, biologically aggressive, carcinomas. Centrosome dysfunction drives the transition from low grade tumors to high grade forms associated with cancer dissemination and death. Briefly stated, centrosome defects are found in a fraction of PIN lesions and low grade tumors, and increase during tumor progression to become ubiquitous in malignant prostate carcinoma. Pericentrin levels are elevated in tumors with centrosome defects, and artificial elevation of pericentrin in cultured cells induces or exacerbates prostate tumor-like features. The oncogenic properties of pericentrin lie within domains that interact with kinases that are themselves implicated in tumorigenesis (PKA, PKC, and others). We recently discovered a novel centrosome gene that induces cell cycle exit when functionally abrogated, suggesting a unique approach to block tumor cell proliferation. This method can be used to induce cell cycle exit of prostate tumor proliferation. Inhibit prostate tumor cell proliferation through prostate-specific targeting and expression of a retrovirus containing a centriolin construct that drives cell cycle exit. For example, one can construct a “double targeting” self-activation replication-defective retroviral vector that has receptors for PSMA and expresses a dominant negative G₀-inducing centriolin construct under transcriptional control of the prostate-specific probasin promotor. The G₀ virus can be specifically targeted with the expression of the G₀ virus to prostate cancer cell lines. The G₀-inducing retrovirus can be specifically targeted to, and arrest, prostate tumor cells in xenographs and in the TRAMP prostate cancer mouse model. One can inhibit prostate tumor cell proliferation through prostate-specific targeting and expression of a retrovirus containing a centriolin construct that drives cell cycle exit. To do this, ones can construct a “double targeting” self-activation replication-defective retroviral vector that has receptors for PSMA and expresses a dominant negative G₀-inducing centriolin construct under transcriptional control of the prostate-specific probasin promotor. Next, one tests the specific targeting and expression of the G₀ virus to prostate cancer cell lines. The G₀-inducing retrovirus can be specifically targeted to, and arrest, prostate tumor cells in xenographs and in the TRAMP prostate cancer mouse model

We have observed centrosome defects in a set of PIN biopsies from patients who proved to have aggressive carcinoma after prostatectomy. The presence of centrosome defects in pre-invasive lesions, and the ability to induce centrosome defects and tumor-like features in prostate cells by overexpressing pericentrin, demonstrates that centrosome defects drive prostate tumorigenesis and accelerate tumor progression. Examination of the PIN biopsies and prostatectomy tissues revealed a correlation between the presence of defective centrosomes in PIN lesions and the subsequent development of aggressive carcinoma.

We have obtained 200 cases of PIN lesions (detected in needle biopsies) that progressed to invasive cancer (detected after prostatectomy) through a collaboration with several institutions, including Walter-Reed Medical Hospital. Biopsies with PIN lesions in which prostatectomy showed only indolent disease have been provided (n=57). Immunohistochemical and immunofluorescence can be used to identify centrosome defects in the PIN lesions and aggressive tumors; we have identified centrosome features that can be analyzed for predictive value (see above). In addition, the level of pericentrin in PIN lesions has predictive power, as we have shown that pericentrin levels are increased in all tumors and that they increase from low to high grade. Centrosome defects can be seen in all PIN lesions from patients who subsequently develop high grade tumors. Centrosomes contribute to changes associated with high grade tumors. This observation has important prognostic value. The current clinical management of patients with “PIN-only” sextant biopsies is controversial because tumor progression from this stage has not been established by other researchers. Centrosome defects in PIN can define patients at high risk of developing high grade prostate carcinoma and assist clinicians in their therapeutic decision. Examination of the above centrosome features and pericentrin levels enables one to identify even subtle changes.

Studies on the histopathology, DNA content, and molecular composition of human material have demonstrated that PIN lesions in proximity to invasive carcinoma are structurally and genetically related to the carcinoma, demonstrating that the invasive component arose from neighboring PIN lesions. Centrosome defects contribute to tumor progression, and such defects are present (or more severe) in PIN lesions adjacent to invasive carcinomas, whereas PIN lesions distant from the tumor, and those adjacent to low grade tumors, may have no centrosome defects. Tissue derived from radical prostatectomies by immunoperoxidase labeling to determine whether centrosome defects are present exclusively (or are more severe) in PIN lesions adjacent to invasive carcinoma can be compared with those more distant from tumor tissue.

### Other Embodiments

A number of embodiments of the invention have been described. Nevertheless, it will be understood that various modifications may be made without departing from the spirit and scope of the invention. Accordingly, other embodiments are within the scope of the following claims.

What is claimed is:

1. A method of predicting the evolution of an in situ lesion in a subject, the method comprising
(a) examining a microtubule organizing center of a cell in a tissue sample from an in situ lesion of a subject,
(b) detecting a centrosome abnormality in the cell, and
(c) determining the degree of severity of any centrosome abnormality detected, wherein the degree of severity of any centrosome abnormality correlates with the probability that the in situ lesion will evolve into high grade invasive cancer.

2. The method of claim 1, wherein the tissue sampled is prostate, breast, uterine cervix, lung, brain, colon, or epithelial.

3. The method of claim 1, wherein any or all of (a), (b), and (c) are automated.

4. A method of predicting cancer in a subject, the method comprising
(a) examining a microtubule organizing center of a cell in a tissue sample from a subject, and
(b) detecting a centrosome abnormality in the cell, wherein the presence of a centrosome abnormality indicates an increased probability that the patient will develop cancer.

5. The method of claim 4, wherein the centrosome abnormality is a diameter of a centrosome greater than twice the diameter of centrosomes present in normal epithelium in the same tissue sample.

6. The method of claim 4, wherein the centrosome abnormality is a centrosome in which the ratio of the centrosome's greatest and smallest diameter exceeds about 2.

7. The method of claim 4, wherein the centrosome abnormality is abnormal shape.

8. The method of claim 4, wherein the centrosome abnormality is the absence of a centrosome.

9. The method of claim 4, wherein the centrosome abnormality is centrosomes that are organized as multiple small dots.

10. The method of claim 4, wherein steps (a) and (b) are repeated for multiple cells, and the centrosome abnormality detected is (1) the presence of more than two centrosomes in more than about 5% of the cells whose microtubule organizing centers are examined or (2) a ratio of centrosomes to nuclei of greater than about 2.5 in the cells examined.

11. The method of claim 4, wherein the centrosome abnormality is an increased level of pericentrin.

12. The method of claim 4, wherein the tissue sampled is uterine cervix, breast, prostate, colon, brain, lung, or epithelial.

13. A method of predicting the degree of aggressiveness of cancer in a patient, the method comprising
(a) examining a microtubule organizing center of a cell in a tissue sample from a precancerous lesion of a patient,
(b) detecting a centrosome abnormality in the cell, and
(c) determining the degree of severity of any centrosome abnormality detected, wherein the degree of severity of any centrosome abnormality correlates directly with the probability that the patient has or will develop aggressive cancer.

14. The method of claim 13, wherein an about 2- to 4-fold increase in the incidence of centrosomal abnormality compared to normal cells correlates with histologic/cytologic grade of cancer.

15. The method of claim 13, wherein the tissue sampled is uterine cervix, breast, prostate, colon, brain, lung, or epithelial.

16. A method of predicting cancer in a subject, the method comprising
(a) examining a mitotic spindle of a cell in a tissue sample from a subject, and
(b) detecting any mitotic spindle abnormality in the cell, wherein detection of a mitotic spindle abnormality indicates an increased probability that the subject has or will develop cancer.

17. The method of claim 16, wherein the tissue sampled is uterine cervix, breast, prostate, colon, brain, lung, or epithelial.

18. A method of predicting cancer in a subject, the method comprising
(a) measuring the level of pericentrin in a cell culture or tissue sample of interest,
(b) comparing the level of pericentrin in (a) to the concentration of pericentrin in a normal, healthy control cell culture or tissue sample, and
(c) predicting an enhanced probability of developing cancer if the level of pericentrin in a cell culture or tissue sample of interest is greater than that in the normal, healthy control cell culture or tissue sample.

19. The method of claim 18, wherein the level of pericentrin in the cell culture or tissue sample of interest is at least about twice the level of pericentrin in the normal, healthy control cell culture or tissue sample.

20. A system for detecting centrosome abnormalities automatically, the system comprising
(a) a cell culture or tissue sample to be examined,
(b) a means for automatically preparing the cell culture or tissue sample for examination,
(c) a high magnification microscope,
(d) an XY stage adapted for holding a plate containing a cell culture or tissue sample and having a means for moving the plate for proper alignment and focusing on the cell culture or tissue sample arrays,
(e) a digital camera,
(f) a light source having optical means for directing excitation light to cell culture or tissue sample arrays and a means for directing fluorescent light emitted from the cells to the digital camera,
(g) a computer means for receiving and processing digital data from the digital camera, wherein the computer means includes a digital frame grabber for receiving the images from the camera, a display for user interaction and display of assay results, digital storage media for data storage and archiving, and a means for control, acquisition, processing, and display of results, and
(h) a computer means for detecting centrosome abnormalities in the cell culture or tissue sample.
